(19) United States
(12) Patent Application Publication
(10) Pub. No.: US 2008/0063560 A1
(43) Pub. Date: Mar. 13, 2008

(54) Ground treatment for reducing microbial content

(75) Inventor: Kenneth R. Code, Edmonton (CA)

Correspondence Address: Mark A. Litman & Associates, P.A.;York Business Center, Suite 205, 3209 West 76th St., Edina, MN, 55435, US

(73) Assignee: BioLargo Life Technologies, Incorporated

(21) Appl. No.: 11/516,958

(22) Filed: Sep. 7, 2006

(51) Int. Cl. A61L 2/16

(52) U.S. Cl. 422/37; 422/29

(57) ABSTRACT

A process reduces the microbial content in land mass by providing molecular iodine in the land mass in a concentration in aqueous material in the land mass of at least 10 parts per million. The molecular iodine may be added in gaseous or liquid or solid state, and may be formed in situ in the land mass using available water in the reaction.

### BACKGROUND OF THE INVENTION

1. Field of the Invention

The present technology relates to the remediation and improvement of land mass that has been contaminated with microbes such as bacteria, viruses, Rickittsiae and fungi. The technology relates to the treatment of ground such as soil and sand with chemicals acting as antimicrobial agents.

2. Background of the Art

Historically, soils contaminated by solvents, oils, waste spillage and similar organic materials through leakage from storage or piping, accidental spills or inadequate disposal have been managed in several ways. Representative of the approaches which have been taken are: (1) excavation and reburial in an approved landfill; (2) soil flushing using recovery and recharge wells; and (3) in-situ biological treatment using supplemental nutrients and possibly supplemental bacteria.

These methods have not been entirely satisfactory for a host of technical, legal and cost reasons. Excavation and reburial of even moderate size tracts of contaminated soil is enormously costly. Moreover, even properly designed and operated state-of-the-art landfills have been found to leak. Soil flushing has serious technical limitations in that very large volumes of water must be flushed through the soil to sufficiently lower contaminant concentrations. In-situ biological treatment also possesses drawbacks, among them its unsuitability in areas with low groundwater and its high cost and the need to introduce new bacteria into an ecological region.

An alternative method of treatment for soils containing easily volatilized contaminants was attempted by the U.S. Environmental Protection Agency (“EPA”) in 1984. The contaminated soil, containing 1,1,1 trichloroethane, trichloroethene, toluene, ethyl benzene and ortho-, meta-, and para-xylene in concentrations from 10,000 to 110,000 ppb., was fed through an asphalt drying unit, also known in the industry as an aggregate dryer or rotary kiln. The drying unit included a liquid propane burner at one end which supplied combustion gases at 375° F. to the interior of the dryer. From exposure to the 375° F. gas, the contaminants were vaporized and the combined mixture of gases passed through a cyclone and bag house for removing particulate emissions, and then discharged directly to the atmosphere. The treated soil was discharged from the dryer, collected and then returned to the site.

Although the foregoing method was reported to be effective in reducing the concentrations of some contaminants by at least 99%, serious drawbacks were evident. Volatile organic compounds (“VOC's”) removed from the soil being treated were discharged to the atmosphere, thus decontaminating one medium, the soil, at the expense of polluting another medium, the air. In order to dilute the concentration of emitted VOC's in the ambient air, and because of local air discharge requirements, the dryer could only be operated at a feed rate of 10-15 tons of soil per hour, rather than the design rate of 100 tons/hour. Localities with more stringent air quality regulations would necessitate an even lower feed rate. Furthermore, the dryer could only be operated in dry weather conditions to prevent the emitted VOC's from being scrubbed from the atmosphere by rainfall, and thus returned to the soil.

For a proper site remediation project, both contaminated soil and contaminated groundwater must be cleaned. The contamination becomes very serious if the groundwater is a drinking water source. About 70 percent of potable water in the U.S.A. is supplied by groundwater. Site contamination, which is a national major concern, is about 71 percent caused by industrial accidents (chemical spills, tank leaks, etc.), 16 percent caused by railroad or truck's chemical accidents, and 13 percent caused by leachates from lagoons or dumpsites.

The primary reason for cleaning soil is public health protection. The primary reasons for treating groundwater are: potable use (39 percent), clean-up of aquifer to prevent spread of contamination (48 percent), and industrial and commercial use (13 percent). In any case, the potentially hazardous VOCs must be removed. Timely clean-up of aquifer to prevent spread of contamination is extremely important because the damage can be beyond repair if the spread of contamination is too wide.

The present technologies for groundwater treatment include: air stripping tower without air emission control, non-regenerative gas phase granular activated carbon, chemical oxidation, non-regenerative liquid phase granular activated carbon, active charcoal fiber, biological processes, ion exchange, ultrafiltration, H₂O₂ treatment, reverse osmosis (RO), ozonation, lime softening, ultraviolet (UV), chemical coagulation, sedimentation, filtration and halogenation (e.g., chlorination, bromination and iodization). Air stripping tower without air emission control is the most common process for VOCs removal, but is not acceptable in many states. Liquid-phase granular activated carbon (GAC) contactor is technically feasible for water purification, but may be economically unfeasible when it is used alone. Chemical oxidation alone or UV alone is not cost-effective for VOCs reduction. Certain chemicals may even give undesirable residuals. Ultrafiltration and RO are excellent post-treatment process, requiring adequate pretreatment for cost reduction. Biological process is very efficient for removal of organic contaminants, but causes air pollution and requires thorough disinfection.

While conventional ozonation, UV, RO and chlorination are all effective disinfection processes, they all require separate reactors.

The story is quite different for soil treatment are quite limited and more narrowly focused because of the complexities in working with masses and volumes of solids. The present technologies for soil cleaning include: surfactant washing, neutralization, solidification, incineration, chemical oxidation, bio-oxidation, lime treatment, venting, and the like.

U.S. Pat. No. 4,715,965 (Sigerson) describes a method for separating volatilizable contaminants from soil by introducing the soil into a rotary aggregate dryer through which a working gas indirectly heated to between 750.degree. F. and 1800.degree. F. is drawn to vaporize the contaminants, and for recovering the contaminants for environmentally sound disposal or recycling by downstream treatment of the effluent drawn off from the dryer. The downstream treatment includes cooling the effluent to condense and precipitate out a substantial portion of the contaminants and passing the effluent through activated carbon.

U.S. Pat. No. 5,552,051 (Wang et al) teaches a biochemical process system and apparatus for removal of toxic volatile organic compounds, volatile inorganic compounds (VICs), heavy metals, and surfactants from contaminated soil, liquid and gas streams, by means of combined washing, pH adjustment, biochemical reaction, gas stripping, scrubbing, adsorption and regeneration is described. This process system involves soil washing with surfactant, soil-water separation, liquid pumping; liquid treatment using an enclosed gas stripping bioreactor and dissolved air flotation technology; gas purification using a foam collector, a wet scrubber and a self-regenerative gas-phase granular activated carbon (GAC) contactor; and recycling of GAC-purified gas for further liquid treatment by gas stripping and biochemical reactions. The process system is extremely cost-effective for removal of both volatile and nonvolatile pollutants from a contaminated site and eliminates the problem of secondary gas contamination caused by conventional air stripping towers.

U.S. Pat. No. 7,033,509 (Klein) discloses an iodine fluid purification process using a source of fluid; means for delivery of iodine to the source of fluid for use in the purification process. The process provides a means for recovery of the iodine and/or iodine and/or other iodine species derived from the iodine, from the fluid.

U.S. Pat. No. 5,176,836 discloses a new and improved water purification process or method by introduction of molecular iodine into the water supply to impart a desired iodine residual wherein the water is passed through an iodinated anion exchange bed wherein the concentration of I.sub.2 in the flowing water gradually decreases and the ion bed is recharged by treatment with an aqueous iodine solution produced by flowing water through a bed of iodine crystals having connections in parallel with the ion exchange bed and activated periodically e.g. by a timer, by measured flow of water or by residual level to recharge the bed. That system provides for long term microbiological control in water suitable for potable activities. The bed of Iodine crystals is provided with connections for flowing water there through to produce a concentrated (substantially saturated) aqueous iodine solution which is passed through the iodinated resin bed to recharge the bed with bound iodine.

The majority of patents relates to the direct or indirect treatment of water to remove microbes. Such disclosures are shown in U.S. Pat. No. 6,863,905; the use of free elemental iodine to kill or inactivate a large range of microbes (bacteria, virus and other pathogens) particularly in protein-containing solutions such as human blood, human plasma or fractions thereof is described in U.S. Pat. Nos. 5,019,495; 5,128,149; 5,128,150; 5,186,945; 5,360,605; 5,370,869; 5,589,072; and 5,609,864; pentavalent iodine-impregnated resins U.S. Pat. No. 5,635,063; provision of potable water U.S. Pat. No. 6,139,731 and U.S. Pat. Nos. 6,071,415; 5,324,438 describes a process for oxidizing a compound comprises contacting the compound with iodide ions and irradiating the iodide ions with UV light of a wavelength sufficient to generate iodine atoms. The compound is then oxidized with the resulting iodine atoms. The iodine atoms are reduced to iodide ions as a result of the oxidation of the compound.

There are not many alternatives available for soil treatment, especially soil treatment for large areas that have been contaminated by microbes, such as beaches and park land. One such treatment is marketed as a proprietary process called “Prolamine Technology.” This technology combines amino acids, or proteins, and polysaccharides, which are basic sugars, to create substances that can address a variety of environmental concerns. Made entirely with GRAS (Generally Regarded as Safe) materials, the products offered by amino solutions are safe to use in soil and water per the standards of the clean air and water act. Amino-Rx—Mold Kill Solution uses a two step approach to kill mold and prevent it from growing back. First, it kills the existing mold by attacking and neutralizing its food source. Then, Amino-Rx forms a protective barrier on the treated surface to prevent the mold from growing back. Amino-Rx will work on most interior and exterior surfaces. Haz-Rx—Hazardous Materials Remediation Solution consists of a third combination of unique ingredients. Designed to remediate hazardous materials contamination, Haz-Rx will break down and neutralize various non-radioactive hazardous materials. If the hazardous materials cannot be neutralized, Haz-Rx will form a protective encapsulation around the materials making them insoluble and preventing any damaging effects.

Phytoremediation is the use of plants and their associated microorganisms for the in situ treatment of contaminated soils, is a steadily emerging technology with potential for the effective and inexpensive cleanup of a broad range of organic and inorganic wastes. Based on a review of the relevant literature, we provide examples of the phytoremediation of petroleum hydrocarbons and discuss the key mechanisms as well as the special considerations involved in petrochemical phytoremediation. The benefits, limitations, and costs of phytoremediation compared to alternative approaches—including natural attenuation, engineering and bioremediation—are well known.

Pesticides can influence soil microbial activity, at times paradoxically. Application of paraquat led to buildup of fungi and bacteria, but reductions in CO₂ production, cellulose degradation, and nitrogenase activity. Sometimes selective destruction of predators and the resultant buildup of their microbial prey can occur. For example, glyphosate or diquat+paraquat application led to the buildup of Gaeumannomyces graminis var. tritici, the causal agent of take-all disease of wheat. Inoculation with untreated soil led to suppression of the pathogen in the treated soil, suggesting the possible role of microbial antagonists.

Nitrification and symbiotic nitrogen fixation are especially sensitive to disruption by pesticides, probably in part due to the small numbers of species involved in these processes. At normal application rates, Amitrole, 2,4-DB, and diallate can inhibit nitrification for at least 8 weeks, whereas atrazine, bromacil, picloram, and simazine can do so for shorter periods. Some degradation products of these substances may also be inhibitory. There are also examples of no effect and (paradoxically) even stimulation of nitrification for some of the pesticides mentioned above. For symbiotic nitrogen fixation, denitrification, and ammonification, there are also cases of inhibition, no effect, and stimulation, but no specifics are recounted. Soil respiration is relatively in-sensitive to pesticide application, but antimicrobials, e.g., fungicides, can suppress it.

It is necessary to provide some significant process for the remediation of microbe-contaminated soil and sand that is effective, fast-acting and economically feasible for large areas.

### SUMMARY OF THE INVENTION

Soil is treated with molecular iodine in vapor or dissolved liquid form to provide a concentration in water or aqueous mass of at least about 10 parts per million. The molecular iodine (as opposed to iodide anion) is provided as a gas or liquid or provided as two reactants that form molecular iodide in the soil, either by using an aqueous carrier, water of hydration or ambient ground water. The source of molecular iodine may be topically applied, ploughed into the soil, mixed into the soil, injected into the soil, sprayed onto the soil, or otherwise applied where desired.

### DETAILED DESCRIPTION OF THE INVENTION

Land mass, such as soil and sand, can be contaminated by microbes in a number of manners. The most common manner of soil contamination is from improper handling or disposal of organic wastes and sewage. Excessive rainfall can also stress sewage systems, causing them to overflow and spill raw sewage over the land. Whatever the source of the microbial contamination, the danger to animal life can persist for extended periods of time and can severely affect both the medical and economic health of an area. It is therefore essential that methods and plans be developed that can treat a wide variety of microbial contaminations, and do so in a rapid manner and at acceptable costs. The problem has been that soil mediation or repair is far more complex and difficult than water purification techniques.

Water can be readily transported through pipes into treatment areas, through filters, or be loaded with chemistry that rapid spreads through the water system to attack microbes. Land mass can not be moved about as readily, and materials added to soil do not disperse as widely as materials added to aqueous systems. Materials added to soil for purposes of microbe reduction or elimination must not persist beyond their useful life and must not contribute a contamination effect themselves.

The technology disclosed herein is based on the discovery that the provision of molecular iodine into microbially contaminated land mass (e.g., soil or sand) can mediate the land mass by killing or at least reducing the concentration of the vast majority of microbes that would ordinarily persist in the land mass.

Land mass (generally soil and/or sand) may become contaminated with any variety of microbes that may be harmful to vegetation or fauna that come into contact with the microbes. The land mass is then treated with molecular iodine in vapor or dissolved liquid form to provide a concentration in water or aqueous mass of at least about 10 parts per million. The molecular iodine (as opposed to iodide anion) is provided as a) a gas, b) liquid or c) provided as two reactants that form molecular iodide (s a gas or into a liquid) in the soil, either by using an aqueous carrier, water of hydration or ambient ground water. The source of molecular iodine may be topically applied, ploughed into the soil, mixed into the soil, injected into the soil, sprayed onto the soil, or otherwise applied where desired. Elemental iodine is a biocidally active form of iodine that has been used as a water disinfectant for almost a century. It is also widely used as a sanitizing compound in the food processing industry. Chlorine solution (especially hypochlorites) have been widely using by growers as a sanitizing wash for many fruits and vegetables. However, the strong oxidizing effect of chlorine in water with a moderate to high organic load results in a number of different complex compounds (trihalomethanes or THM) which can become a significant environmental hazard. There are strong reasons to minimize the excessive use of chlorine in the environment.

One way of providing molecular iodine (I₂) on site, rather than having to find a way of transporting it to a site) is to provide reactants that can readily produce molecular iodine on-site in a controllable reaction. One format of providing the molecular iodine would be through the oxidation-reduction reaction between two salts to produce the molecular iodine. It is a readily controlled environment where the reaction can be performed in an aqueous environment. One reaction that can effect this would be generically described as:

X⁺Y⁻+Z+I−→X^{o}+Z⁺Y⁻+I₂

In this reaction scheme, X is a metal (preferably a multivalent metal and more particularly a divalent metal), Y is an anion (preferably a multivalent anion and more preferably a divalent anion, and an anion having at least two oxygen atoms), Z is an alkali metal or alkaline cation. Examples of X are copper, iron, manganese, lead, nickel, tin, and the like, Y can be sulfate, sulfite, sulfonate, carbonate, phosphate, phosphate, nitrate, nitrie, borate, and the like, and Z can be sodium, lithium, potassium, ammonium, magnesium, aluminum, and the like. One preferred reaction would be:

Cu⁺²SO₄⁻²+K⁺I⁻→Cu^{o}+K₂SO₄+I₂

This reaction takes place readily in an aqueous environment and produces molecular iodine at a controlled rate. The reaction may be used, as intimated above, by either causing the reaction to occur in a container and directing the iodide into the soil (as by gas injection) or by dissolving the molecular iodide and injecting or spraying the dissolved iodide into or onto the soil. The reaction may also be used by dispersing or mixing the two ingredients into the land mass, either with additional water provided, with water of hydration on the first reactant (e.g., X⁺Y⁻.nH₂O, such as CuSO₄.5H₂O) or with ambient water in the land mass. The two reactants may be physically separated from each other before being combined for application or reaction, as in separate pouches or containers. The two reactants may be provided in a solid carrier medium that separates the two reactants until they are in contact with water (as in a soluble carrier such as polyvinyl alcohol, gelatin, amylase, sugars and the like, in pellet or block form). The two reactants may be provided as liquids in separate containers to be mixed immediately before application. The two reactants may be independently coated with a soluble/dispersible coating and the two ingredients kept in a single water-tight container.

If provided in solid form (e.g., pellets, grains, tablets, powder, blocks, etc,), the solid is preferably mixed into the soil rather than merely spread on top of the soil or sand, so as to prevent winds from blowing the solid away. If the solids are sufficiently large (e.g., at least 1.0 mm, preferably at least 2.0 mm in diameter), they can be more safely sprinkled on the surface of the soil or sand without as much concern of being blown away or unevenly distributed by the wind. The solids may be otherwise ploughed into the soil or sand, raked into the soil or sand, injected into the soil or sand, mixed with solid and sand and deposited onto the soil and sand or otherwise securely applied.

It will be apparent to one skilled in the art that there are various reactant chemicals that can be used. The reaction between anhydrous cupric sulfate and potassium iodine to produce iodine is one which is known in the art. Generally two parts (molecular stoiciometry) potassium iodine is required for every one part of anhydrous cupric sulfate to produce the desired reaction. In order to avoid problems in implementing the invention with the chemicals described above, the following matters should be noted. When using container or mixing prior to application, non-ferrous mixing containers and non-ferrous application instruments (or polymer coated ferrous material) should be used in order to avoid galvanic depositing of copper from solution. Application with absorbent and superabsorbent carriers (acrylic polymers, for example) has been found to require an additional amount of cupric sulfate over and above that used for the reaction. The reason for this is believed to be that the substrate has a tendency to sequester multivalent ions. With mixing in the vicinity fo workers, care should be taken to consult safety data sheets relating to iodine gas before experimentation of any magnitude is conducted.

Soil microorganisms tend to congregate at the soil surface in a shallow layer of approximately 10 centimeters in depth. This shallow layer is referenced as either the weathering layer or the plough layer. The large majority of food (leaf fall, plant and animal detritus, etc.) is available at the soil surface. Natural biodegradation end products are fulvic and humic acids which may take up to 25-30 years to biodegrade. Microbial population size bears a direct relationship to the availability of food sources. A distribution of microorganisms may exist in the initial 75 centimeters of a soil profile and may include aerobic bacteria, anaerobic bacteria, actinomycetes, fungi, viruses, rickettsiae and algae. The total aerobic and anaerobic bacteria in the upper 8 cm of soil may be 77-80 percent of the total bacteria found in the 75 cm. profile. 95 percent of all bacteria may be found in the upper 25 cm. of the soil profile. Aerobic bacteria may average between 80-90 percent of the total bacteria for the soil horizons investigated. Thus it is desirable that the gas be provided through the major portions of this depth, e.g., at least to 8-25 centimeters.

Iodine is the preferred sanitizing agent in the food industry as it is acknowledged as a more effective user friendly sanitizing agent than chlorine. In addition, depending upon the concentrations, it is safe, can be effectively used at reduced concentrations (up to ten times less) than chlorine yet with a higher microbial kill rate. Iodine (unlike chlorine) does not produce any harmful substances such as carcinogens, and if nearly all by-products are removed, can produce an environmentally safe waste water. Being a solid at room temperatures and supplied, immersed in water, the potentially harmful effects of exposure to a concentrated sanitizing agent such as chlorine are removed, significantly improving environmental work conditions. Furthermore, iodine is less corrosive than chlorine reducing corrosive effects from the use of a biocide.

A number of United States patents disclose the use of iodine in conjunction with processes for purification of water. For example, U.S. Pat. No. 4,888,118 discloses a water purification process in which the water is passed through a mass of nylon 4 complex with iodine. The treated water is subsequently passed through nylon 4 to remove iodine from the water.

One of the difficulties with the known systems is to maintain an optimum amount of active iodine delivered into the target water supply for the specified purpose. To date there has been no effective system which can effectively and economically guarantee the delivery of exactly the right amount of active iodine at higher levels into the water used to wash produce in the case where iodine is used for food sanitization or into water delivered through reticulation networks, not only to prevent waste of iodine and economic loss but also to ensure that there is an acceptable minimum of active iodine.

Iodine recovery processes are known whose objective is to recover iodine to compensate for gradual reduction of I.sub.2 in the flowing water and to provide a desired iodine residual. The process described in U.S. Pat. No. 5,176,836 is distinguished from previous systems by providing a continuous long term microbiological control process in a water supply particularly in space vehicle applications wherein I.sub.2 is released into the water stream flowing through a suitable anion exchange resin.

U.S. Pat. No. 5,919,374 discloses a method and apparatus for producing bacteria free iodine species containing drinking water for farm animals under continuous dynamic water flow to produce a saturated iodine species containing aqueous solution at a pre selected temperature and blending the saturated solution with a second water flow to produce a diluted iodine species bacterium free aqueous solution.

U.S. Pat. Nos. 4,131,645; 5,356,611; 5,464,603; 5,639,452; 6,139,731; and 6,004,465 disclose prior art processes in which iodine is employed, each of which is incorporated herein by reference. The processes described in those US patents do not teach the use of means to effectively and economically control delivery of iodine in a water stream, nor do they disclose collection and conversion of iodide to iodine species for re use in the process.

Iodinated resin beds are known as a means for recharging a water supply with a minimum amount of active iodine. The recharging is effected by treatment with an aqueous iodine solution produced by flowing water through a bed of iodine crystals. The iodine residual is monitored and the bed recharged where necessary by adjusting the flow rate of water through the bed of iodine crystals. This is an expensive method of monitoring the level of active iodine and the resin rich in bound iodine is very expensive. In addition, the capacity of the resin is limited and reloading techniques in the field would be difficult to maintain in high water flow conditions. Also, this process is best suited to low level (<4 ppm) delivery of active iodine usually in a clean filtered water environment. This is due to the slow dissolving rate of iodine from known iodine beds and the limitation of the release rate and saturation of the anion exchange resins.

An ideal level of active iodine to be maintained in the aqueous content in the soil or sand is in the range of at least or greater then 10 ppm to 25 ppm although some applications may require higher concentrations. When iodine is used in large spill sanitizing applications, it may react with organic matter in which case the active iodine can be reduced to the point where there is little left for microbiological control. If resins (e.g., superabsorbing polymers) are used to deliver active iodine, this could necessitate continual monitoring of iodine concentration. It is expensive to use resin in large areas of soil, so it is likely that this mode of delivery would be used in more localized areas. Saturation of resin with 46% weight Iodine will produce around 4 ppm active iodine release, which is insufficient alone, but with the reactive mixture, higher concentrations of molecular iodine can be provided. A controlled iodine delivery process would be one in which the level of iodine can be maintained at a predetermined optimum level and without constant manual intervention and monitoring.

The process technology of the present disclosure may be practiced in a number of formats, such as a process for reducing the microbial content in land mass by providing molecular iodine in the land mass in a concentration in aqueous material in the land mass of at least 10 parts per million. The aqueous material should have a concentration of at least 10 parts per million is applied to the land mass. Specific formats include two reactants are added to the land mass and the two reactants react in the presence of water to generate a concentration of at least 10 parts per million in the water of the molecular iodine, especially where the two reactants are a) mixed with the land mass and at least some of the water present is ambient water; b) mixed with the land mass and at least some of the water present is water of hydration of one of the two reactants; c) mixed with the land mass and at least some of the water present is applied to the land mass at about the same time as the application of the two reactants; d) mixed with the land mass and at least one of the two reactants is coated to prevent premature reaction with water or another reactant. The process is particularly useful on recently contaminated sites, especially where the contaminant microbes reside in the top 25 cm of the soil such as where the land mass is sand at a site where organic waste matter has contaminated the san with microbes.

Among the ways of applying the molecular iodine are at least where molecular iodine gas is injected into the land mass; where the molecular iodine gas is generated in a closed container and injected into the land mass; where the land mass is physically disturbed to assist mixing of molecular iodine into the land mass; where physical disturbance comprises plowing of the land mass; and where solid reactant material to generate the molecular iodine is deposited in the land mass by the physical disturbance. The process may use the above reaction to form the molecular iodine represented by

XY+ZI→X^{o}+ZY+I₂

wherein X is a metal, Y is an anion, Z is an alkali metal or alkaline cation, or where X is a multivalent metal, Y is a multivalent anion, and Z is an alkali metal or alkaline cation, and is preferably represented by

Cu⁺²SO₄⁻²+K⁺I⁻→Cu^{o}+K₂SO₄+I₂.

The process may be performed where the two reactants are carried in a superabsorbent polymer. The solids carriers for the two reactants may also include compositions of the present that comprise superabsorbent or non-superabsorbent polymers, natural products (e.g., papers, cellulosic solids, water-insoluble porous materials which absorb or adsorb the film-forming material within the structure, water-soluble porous materials which absorb or adsorb the film-forming material within the structure, porous containers which merely slowly release a volume of the film-forming material, porous containers which both dissolve and physically release volumes of the film-forming composition through pores, and the like. In general, selection of an effective application rate can depend on habitat depth, surface debris, emergent and surface vegetation, organic matter, microbial and algal concentration, the specific target species, and the developmental stage of the target species. Superabsorbent polymers are described, by way of non-limiting examples in U.S. Pat. Nos. 6,403,674; 4,731,391. Superabsorbent polymers, including starch graft co-polymers, are known in the art. See, for example, those described in U.S. Pat. Nos. 4,375,535 and 4,497,930 (incorporated herein by reference), which have disclosed uses as adhesives, flocculants, sizes, water-retaining materials for agriculture and water-absorbing materials for sanitary materials. However, the spectrum of advantages attendant the use of superabsorbent polymers in solid and flowable terrestrial insecticidal, pesticidal or insecticidal/pesticidal delivery compositions have gone unrecognized.

The superabsorbent polymers of the present invention are synthetic organic polymers which are solid and hydrophilic, absorbing over 100 times their weight in water. These superabsorbent polymers are typically in a powder, granule, extruded, or flake form, adapted to be blended and/or agglomerated into any shape or form.

The superabsorbent polymers may be, for example, acrylamide alkali metal acrylate co-polymers; propenenitrile homo-polymers, hydrolyzed, alkali metal salts; polymers of propenamide and propenoic acid, alkali metal salts; hydrolyzed acrylonitrile co-polymers, and starch graft co-polymers and ter-polymers thereof. All of these are designed to be hydrophilic, absorbing over 100 times their weight in water. The resulting hydrophilic polymers can absorb from over one hundred to greater than about 5000, more typically around 500 to about 1,000, times their own weight in water (measured using distilled water, pH 7.5, 25, 760 mm Hg. absorption within about 30 seconds). However, the absorption or swelling capacity and absorption or swelling time typically varies with each specific superabsorbent polymer.

One class of superabsorbent polymers include combinations of a starch and organic monomers, oligomers, polymers, co-polymers or ter-polymers. They may be manufactured in a variety of ways, for example, the methods described in U.S. Pat. Nos. 4,375,535 and 4,497,930, and can be, for example, the product of grafting corn starch (amylopectin) with acrylonitrile (an acrylic monomer or oligomer). A second class of superabsorbent polymers includes combinations of acrylamide and acrylate polymers, co-polymers and ter-polymers.

All references cited herein are incorporated by reference in their entirety.

What is claimed:

1. A process for reducing the microbial content in land mass comprising providing molecular iodine in the land mass in a concentration in aqueous material in the land mass of at least 10 parts per million.

2. The process of claim 1 wherein an aqueous material having a concentration of at least 10 parts per million is applied to the land mass.

3. The process of claim 1 wherein two reactants are added to the land mass and the two reactants react in the presence of water to generate a concentration of at least 10 parts per million in the water of the molecular iodine.

4. The process of claim 3 wherein the two reactants are mixed with the land mass and at least some of the water present is ambient water.

5. The process of claim 3 wherein the two reactants are mixed with the land mass and at least some of the water present is water of hydration of one of the two reactants.

6. The process of claim 3 wherein the two reactants are mixed with the land mass and at least some of the water present is applied to the land mass at about the same time as the application of the two reactants.

7. The process of claim 3 wherein the two reactants are mixed with the land mass and at least one of the two reactants is coated to prevent premature reaction with water or another reactant.

8. The process of claim 1 wherein the land mass is sand at a site where organic waste matter has contaminated the san with microbes.

9. The process of claim 1 wherein molecular iodine gas is injected into the land mass.

10. The process of claim 10 wherein the molecular iodine gas is generated in a closed container and injected into the land mass.

11. The process of claim 1 wherein the land mass is physically disturbed to assist mixing of molecular iodine into the land mass.

12. The process of claim 11 wherein physical disturbance comprises plowing of the land mass.

13. The process of claim 11 wherein solid reactant material to generate the molecular iodine is deposited in the land mass by the physical disturbance.

14. The process of claim 3 wherein the reaction to form the molecular iodine is represented by
XY+ZI→X^{o}+ZY+I₂
wherein X is a metal, Y is an anion, Z is an alkali metal or alkaline cation.

15. The process of claim 3 wherein the reaction to form the molecular iodine is represented by
XY+ZI→X^{o}+ZY+I₂
wherein, X is a multivalent metal, Y is a multivalent anion, and Z is an alkali metal or alkaline cation.

16. The process of claim 3 wherein the reaction to form the molecular iodine is represented by
Cu⁺²SO₄⁻²+K⁺I⁻→Cu^{o}+K₂SO₄+I₂.

17. The process of claim 3 wherein the two reactants are carried in a superabsorbent polymer.
